SEQUENCE LISTING (2 sequences)

SEQ ID NO: 1 - protein, 16 aa, Leishmania sp.
  MISC_FEATURE (4)..(4): Xaa is Cys or Ser
AARXARXREGYXXTDE

SEQ ID NO: 2 - protein, 16 aa, Leishmania sp.
  MISC_FEATURE (3)..(4): Xaa is Ser or Cys
AAXXTPXPGXGXEVDG

(19) United States
(12) Patent Application Publication
(10) Pub. No.: US 2005/0079186 A1
(43) Pub. Date: Apr. 14, 2005

(54) Therapeutic vaccine peptide complex for preventing and treating disorders in mammals

(75) Inventors: Gerard Papierok, Hyeres (FR); Serge Vicens, Gignac la Nerthe (FR)

Correspondence Address: HARRISON & EGBERT, 412 MAIN STREET, 7TH FLOOR, HOUSTON, TX, 77002, US

(73) Assignee: ORIDAN, Inc., Wilmington, DE (US)

(21) Appl. No.: 10/489,586

(22) PCT Filed: Sep. 13, 2002
(86) PCT No.: PCT/FR02/03134

(30) Foreign Application Priority Data
Sep. 14, 2001 (FR) 01/11942

(51) Int. Cl. A61K 39/00

(52) U.S. Cl. 424/185.1; 514/12; 514/14

(57) ABSTRACT

A therapeutic vaccine peptide complex for preventing or treating disorders in mammals and particularly in humans, in Canidae, Felidae and Equidae whose protective immunity depends on the stimulation of type Th1 lymphocytes and in particular a delayed state of hypersensitivity. The vaccine peptide complex contains the following amino acid sequence (A16E): A-A-R-S-A-R-S-R-E-G-Y-S-L-T-D-E sequence wherein L can be substituted by I, and S by C, and/or the following amino acid sequence (A16G): A-A-S-S-T-P-S-P-G-S-G-C-E-V-D-G sequence wherein C can be substituted by S, and S by C, and an adjuvant which preferably induces a cell-mediated response.

### RELATED U.S. APPLICATIONS

Not applicable.

### STATEMENT REGARDING FEDERALLY SPONSORED RESEARCH OR DEVELOPMENT

Not applicable.

### REFERENCE TO MICROFICHE APPENDIX

Not applicable.

### FIELD OF THE INVENTION

Therapeutic peptide vaccine complex designed for the prevention and treatment of conditions in mammals and in particular, in humans, canines, felidae, and equidae whose protective immunity depends on the stimulation of T lymphocytes of the Th1 type and notably of a state of hypersensitivity of the retarded type.

Therapeutic peptide vaccine complex also designed for diagnosis of cell-mediated immunity depending on the T lymphocytes of the Th1 type using various “in vivo” and “in vitro” methods.

### BACKGROUND OF THE INVENTION

During the immune response, the T lymphocyte comprises an important source of cytokines. Their production is induced after stimulation in a specific manner in the presence of antigens or in a non-specific manner in the presence of mitogens (concanaviline A, phytohemagglutinin).

If it is clearly established that the T lymphocytes, and among them, especially the CD4+ lymphocytes, represent the main source of cytokines, the T sub-populations involved in this phenomenon are varied and seem to vary as a function of the stimulus.

It is possible to divide, in a very simplified way, the immune responses into two large qualitatively distinct categories, humoral responses which bring into play the production of antibodies by the B lymphocytes, and cellular responses (retarded hypersensitivity reaction, cytotoxic reaction), for which the effective cells are the T lymphocytes. It seems that in the majority of experimental models and clinical situations studied, the cytokines having a lymphocytary origin are essentially produced by the auxiliary T cells CD4+ (or helpers), whose role is to modulate or regulate the humoral and cellular immunity. These auxiliary T cells recognize the antigen in combination with the class II molecules of the major histocompatibility complex. A major concept emerged in 1985 when T. Mosmann and R. Coffman proposed that the T lymphocytary cells CD4+ expressing auxiliary functions were in fact heterogens. Thus, using a study of the lymphocytary clones T CD4+ of mice, cultivated over the long term, the authors described the existence of two major sub-populations that can be distinguished by their profile of secretion of cytokines, i.e. Th₁ cells (for T helpers of type 1) and Th₂ cells (for T helpers of type 2).

The example of leishmaniasis will be used, which is a parasitic infection endemic, or even epidemic, in tropical and subtropical regions of the world. The leishmania, flagellate protozoa of the family of trypsanosomatidae and of the genus Leishmania, are the pathogenic agents responsible for these diseases.

Numerous studies concerning the immune responses during experimental murine leishmaniases have led to the demonstration of the predominant role of cell-mediated immunity and the existence of a duality of the immunological response. There are fundamentally two types of responses against leishmanias: one is described by the “sensitivity”, the other is described by the “resistance”. The different subpopulations of T lymphocytes (CD4+) limit or exacerbate the infection by means of the lymphokines they secrete. It has thus been demonstrated that the subpopulation of auxiliary T lymphocytes of the Th1 type (producer of interferon gamma and interleukine 2) was capable of eliminating the amastigote intracellular forms by means of the activation of macrophages (Reiner S. L et al., Annu Rev Immunol, 1995, 13, 151-177. Review). Conversely, the subpopulation of auxiliary T lymphocytes of the Th2 type (producer of interleukine 4) is responsible for exacerbating the disease.

In humans, certain facts are comparable by nature. In the dog (natural “reservoir” receptive host in the life cycle of L. infantum), the duality of the immunological response is likely. Only one study led by Pinelli et al. (Infect. immun., 62:229, 1994) on experimental animals naturally infected by L. infantum, made it possible to show that the asymptomatism of the dog (clinical state frequently encountered) is accompanied by the absence of a humoral response and by the development of a cell-mediated immunity of the Th1 type with a hypersensitivity reaction of the positive retarded type and elevated rates of interleukine 2 and TNF-α circulating in the biological liquids.

A good vaccine candidate must thus match one or more strongly immunogenic parasitic antigens capable either of blocking the differentiation of the Th2 lymphocytes (Gurunathan S et al., J. Exp Med, 1997 Oct. 6, 186, 1137-1147) (mode of intervention comparable to “desensitization” treatments currently practiced in cases of allergy), or promoting the emergence of the Th1 lymphocytes ensuring the implementation of a protective immunity.

Planning to vaccinate against leishmanias is still problematic today. The attempts have been numerous, but the results are weak and/or contradictory. It can be cited that the use of living parasites, irradiated parasites, and completely killed parasites (Moreau Y et coll., 1994, Médecine et Armées, 22, 1, 89-93) which have given variable levels of protection among mice and humans.

In the 1980s, purified extracts of parasitic antigens were used in dogs in inducing an exacerbation of the disease: LIF2 fraction and anti-idiotypical vaccine from Dr. Montjour's team. (CHAUVY, J “Immunotherapy trials on a canine population in an endemic zone of leishmania” thesis no. 36.1993-OGUNKOLADE B.W. et coll. Vet Parasitol.), GP63 and lipophosphoglucane (MOREAU Y et coll, Médecine et Armées, 1994, 22, 1, 89-93) have not produced a satisfactory result. Currently, several molecules are in trials and a final result is pending. It can be cited that the heat shock protein HSP83 of Leishmania major which stimulates the Th1 method and the protein DP72 (JAFFE. C et al, J of Immunol, 1990, 144, 699-706). However, none of the current immunization protocols make it possible to obtain a sufficient level of protection or in any case, it is not reproducible.

To date, no work has been done with synthetic peptides.

### BRIEF SUMMARY OF THE INVENTION

The present invention comprises an immunomodulator complex that uses one or two peptides with an adjuvant inducing either an immunostimulation of the lymphocytary system T of the type Th1 in a reproducible manner, or an immunomodulation of lymphocytes of the type Th2 into a type Th1.

It is important to emphasize two essential facts. Firstly, the Th1 and Th2 cells exert, by means of certain cytokines that they produce, a reciprocal counter-regulation effect, which explains why in the majority of these cases, an alternation of the cell-mediated and humoral responses is observed. Thus, the immune responses that favor a reaction of the T lymphocytes of the Th1 type, i.e. with organisms inducing an immunity depending on the T lymphocytes, are frequently associated with a low humoral response; this is the case, for example, in mycobacterial infections.

Conversely, a response that favors the production of antibodies will be most often associated either with the participation of the T lymphocytes of the Th2 type or with a relative deficiency of the specific cellular immunity; this is the case of visceral leishmaniasis. The second fundamental circumstance is that lymphocytes fully differentiated into cells of the Th1 and Th2 type are not pre-existing in the non-sensitized simple host. During a physiological immune response, after which more often than not the antigens are rapidly eliminated, the activated CD4+ cells, rather than being expressed either by a Th1 type phenotype or by a Th2 type phenotype, usually secrete cytokines specific to the Th1 and Th2 type. This explains why in humans, the demonstration of the existence of a Th1/Th2 dichotomy necessitated the study of the cytokinic system coming from lymphocytes not from normal subjects normally immunized, but from patients having chronic diseases such as parasitic infections, allergies, or auto-immune diseases.

The polarization of the immune responses into a Th1 or Th2 phenotype has been associated with numerous pathological situations.

For infections by leishmania, Typanozoma, Candida, and other intracellular organisms such as Mycobacterium and Listeria, a Th1 response correlates to resistance to the pathogen. On the other hand, certain conditions such as canine atopic dermatitis, allergies and asthma lead to the exacerbation of a response of the Th2 type. The recourse to immunostimulants which permit the passage from a Th2 response to a Th1 response, thus the passage from a state of immediate hypersensitivity to a state of hypersensitivity of the retarded type, must lead to a recovery.

It can be considered that the example of asthma, for which the medical community is worrying about the increasing number of individuals who suffer from it: approximately 200 million people in the world, and according to the World Health Organization, 180,000 of them died in 1997. In France, 2,000 people die from asthma every year.

Asthma is a chronic inflammatory condition of the respiratory tracts where numerous cells of the immune system are intervening.

The disease becomes chronic because of the quasi-permanent accumulation of inflammation mediators, as well as the quasi-incessant recruitment of destructive polynuclear eosinophils: these cells accumulate in the bronchial mucous membrane and release basic proteins that destroy the bronchial epithelium. The damage caused by the basic proteins leads to a hyperactivity of the bronchial tubes: the nerve endings that innervate the bronchial tubes are exposed and are no longer protected from outside attacks.

It can be recalled that the activated T lymphocytes release notably interleukine 4, which starts the production of the immunoglobulins E. This “allergic orientation”, again called method Th2, conflicts with the anti-infectious reactions of the Th1 method: in the context of the fight against a bacteria (the bacillis of tuberculosis, for example), the activated T lymphocytes release interleukine 2 and gamma interferon. The gamma interferon activates the cells which have the antigen, and which, in turn, produce interleukine 12. Thus, interleukine 4 orients the immune reactions towards the allergic reactions, while gamma interferon seems to promote the reactions of the defensive type.

And yet this orientation of the immune system will be set very early, in the very young infant. In a newborn baby exposed to a pathogenic agent, the immune system becomes oriented to the defensive method: it is the T lymphocytes, producers of interleukine 2 and gamma interferon, which are activated first and will stay. These children will have less allergic risks.

On the other hand, newborn babies raised in a sterile setting have risks of being confronted with an environment high in allergens and low in pathogenic micro-organisms.

If this is the case, the still immature immune system will activate the T lymphocytes which secrete interleukine 4, giving the immune system an allergic orientation, which favors asthma. This hypothesis, coming from the work of Tim Mosman and Robert Coffann, from the Institute of Molecular and Cellular Biology DNAX, in Palo Alto, would explain the increase in the prevalence of the asthmatic illness in industrial countries.

A reorientation of the immune reactions of sensitive persons, by limiting the allergic Th2 method of immune response, to an activation of the Th1 method would be a possibility for the treatment of asthma.

It can be extended that this phenomenon to other forms of immediate hypersensitivity.

Immediate hypersensitivity is the most frequent form of allergy and results from the synthesis of immunoglobulins E (IgE), antibodies specific to the allergens from the environment. These immunoglobulins E become attached to the cells whose mastocytes are the key elements. The contact of the allergen and the immunoglobulins E activates the mastocytes which release the mediators of the inflammation: this is degranulation of the mastocytes. Grouped under the name of anaphylaxia and atopic diseases are all clinical manifestations linked to the phenomena of immediate hypersensitivity which result from the production of immunoglobulins E, the great culprits of the allergy. However, anaphylaxia corresponds to a physiopathological mechanism independent of any hereditary factor (it is, for example, the anaphylactic shock due to hymenopteran venoms), while atopia is the expression of a particular genetic aptitude to produce immunoglobulins E in excess directed against various natural substances of the atmospheric environment (pollens, molds, pollutants), household environment (dust mites, cockroaches, mammals) or professional environment, and also against foods; this results in respiratory allergies (asthma, rhinites), cutaneous allergies (urticaria, atopic eczema), ophthalmic allergies and digestive allergies.

In these pathologies, two points must be mentioned:
1—the series of events initiated by the attachment of the allergen complex—IgE on different cellular populations and notably on mastocytes
2—the role of the cytokines which, by the intermediary of the synthesis of the IgE, participate in the sensitization phase, on the one hand, and in the perpetuation of the allergic reaction on the other hand.

Today, treatments that confine the syndromes having an allergic origin comprise a part of the anti-inflammatory arsenal (anti-histamine and corticoids). The predominant place taken by the cytokines in the fundamental mechanisms of immediate hypersensitivity opens prospects for new targeted therapies: it is hoped notably to make the Th1/Th2 equilibrium swing in favor of the Th1 method and to modulate the cellular signalization induced by the cytokines when they become attached to their receptors located on the lymphocytes, but also on the mastocytes and on the eosinophils.

### DETAILED DESCRIPTION OF THE INVENTION

The present invention relates to a vaccine complex comprising 2 peptides named A16E and A16G and/or their derivatives, specific to the parasite Leishmania, combined with an adjuvant. The invention also involves the use of these fragments and the adjuvant as diagnostic reagents in vitro and in vivo, and as reagents that induce a stimulation of the T lymphocytes of the Th1 type ensuring an inhibition of the development of the lymphocytes of the Th2 type, notably for the prevention and the treatment of conditions linked to a condition of immediate hypersensitivity, i.e. an immune condition of the Th2 type.

The two peptides A16E and A16G have the following sequences of amino acids:

| | |
|---|---|
| A16E: | (16 amino acids): |
| | A-A-R-S-A-R-S-R-E-G-Y-S-L-T-D-E |
| A16G: | (16 amino acids): |
| | A-A-S-S-T-P-S-P-G-S-G-C-E-V-D-G |

In the peptide compound A16E, L can be replaced by I, and S by C.

In the peptide compound A16G, C can be replaced by S, and S by C.

The peptide compound according to the invention can also be comprised of possible derivatives of the peptides A16E and A16G, these derivatives comprising at least five adjoining amino acids taken in the sequences A16E or A16G. In this case, the derivatives used will be, preferably, the derivative E-G-Y-S-L taken in the sequence A16E, and the derivative S-G-C-E-V taken in the sequence A16G. The other possible derivatives are:

```
A-A-R-S-A
A-A-R-C-A
    A-R-S-A-R
    A-R-C-A-R
        R-S-A-R-S
        R-C-A-R-C
        R-C-A-R-S
        R-S-A-R-C
            S-A-R-S-R
            C-A-R-C-R
            C-A-R-S-R
            S-A-R-C-R
                A-R-S-R-E
                A-R-C-R-E
                    R-S-R-E-G
                    R-C-R-E-G
                        S-R-E-G-Y
                        C-R-E-G-Y
                            R-E-G-Y-S
                            R-E-G-Y-C
                                E-G-Y-S-I
                                E-G-Y-C-I
                                E-G-Y-C-L
                                    G-Y-S-L-T
                                    G-Y-C-L-T
                                    G-Y-S-I-T
                                    G-Y-C-I-T
                                        Y-S-L-T-D
                                        Y-C-L-T-D
                                        Y-S-I-T-D
                                        Y-C-I-T-D
                                            S-L-T-D-E
                                            C-L-T-D-E
                                            S-I-T-D-E
                                            C-I-T-D-E
A-A-S-S-T
A-A-C-C-T
A-A-C-S-T
A-A-S-C-T
    A-C-C-T-P
    A-C-S-T-P
    A-S-S-T-P
    A-S-C-T-P
        S-S-T-P-S
        C-C-T-P-C
        C-C-T-P-S
            C-S-T-P-C
            C-S-T-P-S
            S-C-T-P-C
            S-C-T-P-S
            S-S-T-P-C
                S-T-P-S-P
                S-T-P-C-P
                C-T-P-S-P
                C-T-P-C-P
                    T-P-S-P-G
                    T-P-C-P-G
                        P-S-P-G-S
                        P-C-P-G-S
                        P-S-P-G-C
                        P-C-P-G-C
                            S-P-G-S-G
                            S-P-G-C-G
                                C-P-G-S-G
                                C-P-G-C-G
                                    P-G-S-G-C
                                    P-G-C-G-C
                                    P-G-C-G-S
                                    P-G-S-G-S
                                        G-S-G-C-E
                                        G-C-G-C-E
                                        G-C-G-S-E
                                        G-S-G-S-E
                                            C-G-C-E-V
                                            C-G-S-E-V
                                            S-G-S-E-V
                                                G-C-E-V-D
                                                G-S-E-V-D
                                                    C-E-V-D-G
                                                    S-E-V-D-G
```

The peptide compound according to the invention can also be included in any polypeptide.

The peptides obtained by synthesis and their derivatives are constructed in octopus or are made immunogenic by connections to the carriers (large molecules of the KLH type), and are administered to mammals in the presence of an adjuvant, preferably muramyl dipeptide (MDP): this combination comprises the therapeutic peptide vaccine complex.

In a preferred manner, the protein/adjuvant ratio is comprised between 1/0.1 and 1/6.

In the case of an infection by leishmaniasis, the studies done in dogs have made it possible to determine the optimal vaccine dose to be 100 μg of muramyl dipeptide for 50 μg of injected proteins. However, the beginning of a response is observed starting at 30 μg of injected proteins.

The mechanism of specific action of the peptide complex obtained according to the invention is verified using traditional methods that allow the dosage of peptides, their identification, and using more specific methods that show that the innovative peptide complex takes effect either by immunostimulation of the lymphocyte system of the Th1 type, or by immunomodulation of a Th2 type towards a Th1 type.

For each mammal studied (dog, for example), a serologic analysis is done with the peptides A16E and/or A16G.

A parasitological exam was done from the sample taken directly from the candidate studied, a dog, for example.

A smear of a puncture of the bone marrow is made on a slide. This smear, fixed once by methanol, is stained with May-Grünwald-Geimsa and observed by an immersion microscope (x 1000).

Samples of the bone marrow are brought under cultivation in the biphase cultivation medium NNN (Novy and Mac Neal, 1904, 4. Infec. Dis., 1:1-30), of which RPMI 1640 supplemented with 20% decomplemented fetal calf serum constitutes the liquid phase. The blind subculturings were done every four to six days. The cultures are regularly observed in a photon microscope (×400) for 20 min.

The parasitemy was quantified as follows:

| | |
|---|---|
| +/− | elongated refractive immobile forms; |
| + | 1 to 5 mobile promastigote forms/field; |
| ++ | >5 mobile promastigote forms/field; |
| +++ | culture at confluence. |

Evidence of the involvement of a cell-mediated immunity of the Th1 type:

Leishmanias of the promastigote forms are cultivated in the standard culture media. Parasites are harvested from the end of the exponential phase (6-7 days). The parasitic residue is washed three times by centrifugation (2500 g, 15 nm, 4° C.) in a PBS buffer.

After having verified the viability of the parasites using a vital stain (Trypan Blue), a suspension containing 2×10′ parasites per ml is inactivated in a PBS buffer containing 0.01% merthiolate (Pinelli et al., 1994, Infect. Immun., 62: 229-235). This constitutes the leishmanias for the intradermoreaction test (IDR).

The study of the immune response of the Th1 type that follows was performed on dogs.

Dogs are placed in lateral decubitus and a delicate and non-irritating shearing is done on the thoracic zone approximately 5 cm by 10 cm behind the elbow. Four circles 10 mm in diameter are marked using a felt-tip pen.

Into the center of the circles, 0.1 ml of solution is injected in an intradermo-injection. Two circles receive the solution of leishmanias and the 2 other circles receive the saline methiolated solution in the negative control. The reading of the Intra Dermo Reaction (IDR) is done 48 hours later using an allergologic gauge.

The test is considered positive if the mean of the two observed induration diameters is greater than or equal to 5 mm. The observation of an erythema without induration will be considered to be a negative test (Pinelli et al., 1994, Infect. Immun., 62: 229-235; Marty et al., 1994, Trans. Roy. Soc. Trop. Med. Hyg., 88, 658-659).

The IDRs can also be performed with a solution of peptides A16E and A16G or their derivatives and MDP, for example, in proportions of 10 μg of peptides for 20 μg of MDP.

The present invention thus also involves a product for in vivo diagnosis which reveals a retarded hypersensitivity immune condition of the Th1 type using peptides A16E and A16G and their derivatives in an intra-dermo reaction in mammals.

One can also carry out dosage of nitrogen monoxide (NO) to know the destructive activity of the monocytes against the Leishmanias. The synthesis of NO by the monocytes is in fact a sign of the destruction of the leishmanias by the monocytes having been activated by the cytokines of the interferon gamma type (IFNγ).

NO has a high chemical reactivity. In the presence of water and oxygen, this molecule is rapidly oxidized in a stochiometric manner and thus forms the nitrites (NO2-) according to the reaction: 4NO°+O₂+2H₂O-4NO₂⁻+4H^{+.}

The nitrites accumulate in the media and are easily detectable chemically by the Griess method.

To 50 μl of supernatant to be tested, 60 μl of 60 μl of Griess B (N-(1-napthtyl)ethyl-enediamine 0.3%) is added. The colorimetric reaction develops in the dark for 2 minutes. The optical densities obtained at 540 nm are corrected by the subtraction of the OD obtained on the wells containing only the culture medium.

The values obtained are recorded on a calibration curve (OD=f(NO₂) made from the known concentrations of NO₂⁻.

For this test, the monocytes and lymphocytes are isolated from the venous blood of dogs. The monocytes are brought under cultivation for 3 days at the rate of 10⁵ cells per well in the culture chambers (Labtek) in a medium RPMI 1640 complete (containing 25 mM HEPES, 2 mM L-glutamine, 100 U penicillin per ml) at 37° C. in a humid atmosphere containing 5% CO₂. After 3 days of cultivation, the macrophages are washed in RPMI complete medium, supplemented with fresh medium. The cells are put in incubation either alone, or in the presence of 5 μg of peptides, or in the presence of autolog lymphocytes.

When they are used, the lymphocytes cultivated separately are washed, counted, and added to the macrophages in the ratio of 2 lymphocytes per macrophage.

Next, a test is done on the lymphocyte proliferation.

The peripheral blood mononuclear cells (PBMC) of dogs are separated on the Ficoll gradient (density 1.078) by centrifugation at 800 g for 20 mn at ambient temperature. These cells are brought under cultivation on a plate having 96 wells at a concentration of 2.10⁵ cells per well in the presence of 2 μg per ml of Concanavalin A (Sigma), 5 μg per ml of ES P (excretion—secretion promastigotes) or 20 ml of supernatants of culture harvested in the stationary phase of growth of promastigotes (SP) per well, and in the absence of any additive in a volume of 200 ml of the medium RPMI 1640 containing 5% decomplemented fetal calf serum, 2 mM of L-glutamine, 100 U of penicillin per ml, 100 mg of streptomycin per ml. The optimal antigen and mitogen concentrations have been determined in prior experiments. The PBMCs are incubated for 72 hours in a humid atmosphere at 37° C. in the presence of 5% CO₂ then for 20 hours with 0.5 μCi of ³H thymidine. The cells are harvested over a filter and the incorporation of the radioactivity is determined by counting in a scintillating liquid (β-counter). All of the tests are done in triplicate.

A more rapid and more sensitive immunohistochemical method using BrdU (5-bromo-2′-desoxyuridine), a structural analog of thymidine, is also used to measure the cellular proliferation (BrdU, cell proliferation detection kit III, Boehringer Mannheim, Germany). In our experiments, the BrdU is added for 18 hours after 72 hours of incubation. The cells which have incorporated the BrdU in their ADN are easily detectable in the presence of a monoclonal antibody directed against the BrdU.

The proliferative responses are expressed in stimulation indices that represent the ratio of the average proliferation after stimulation to the mean proliferation in the absence of antigen.

The lymphocytary proliferation has also been estimated by visual readings in a photon microscope (−: negative; +/−: slight proliferation; +: little proliferation less than 5 points per microscopic field; ++: mean proliferation greater than 5 points; +++: strong proliferation).

In parallel to the study of the specific activation of the T lymphocytes the Th1 type by intradermoreaction, dosage of the NO (NO is synthesized by monocytes activated by the cytokines of the T cells of the Th1 type) and lymphocyte proliferation, a serological monitoring by traditional immunofluorescence using strips coated with the promastigotes (serological reference method for canine leishmaniasis) is done.

For the procedure of the studies, other specific techniques were used.

Method of Infectious Examination:

The infectious examination consists in intravenously injecting 106 treated promastigotes in metacyclic phase in the complement of a healthy dog and 5.106 peritoneal macrophages of a healthy dog, infected in vitro by the amastigotes.

The promastigotes and infected macrophages are diluted in sterile physiological serum for a final volume of 1.5 ml. This mixture is made just prior to injection.

Detection of immunoglobulins of the isotype IgG2 of dogs, specifically of the peptides A16E and A16G and their derivatives:

This detection is done by the ELISA method according to the microtitration technique of Kweider et al (J. Immunol. 1987, 138, 299) using a bonded anti IgG2. For this method, the peptides are biotinylated prior to coating on microplates. This connection to the large molecules of the biotin type notably has the effect of making the peptides A16E or A16G or their derivatives more antigenic.

The peptides can also be linked with glutaraldehyde or combined with the bodies of polylysine (presentation of the OCTOPUS type, for example).

The innovative character of the peptide complex according to the invention does not reside solely in the induction of a specific cellular response of the Th1 type but also in the production of low rates of specific immunoglobulins of the isotype IgG such as the IgG2 in dogs. These specific IgGs can be detected by various in vitro methods, for example: ELISA, DOT BLOT, WESTERN BLOT, IMMUNOCHROMATOGRAPHY, LATEX and any other in vitro method that involves intervention of a conjugate system or other systems for display of the Ag-Ac reaction. For example, an ELISA system can be used on a plastic support, and a WESTERN BLOT system can be used on nitrocellulose membranes or other polymers involving the intervention of a enzyme conjugate. Latex supports can also be used. The A16E, A16G peptides and their derivatives can also be bonded, for example, to radio-isotopes, fluorescent molecules, luminescent molecules, or colored particles.

In fact, certain preliminary work among humans (KAWANO. P et al, Parasite Immunol, 195, 17, 451-458) and in dogs (NIETO C. G et al, Vet Immunol and Immunopathology, 1999, 67, 117-130) shows that the IgG isotypes would be markers of the immunitary dichotomy Th1/Th2. More specifically, a dog suffering from leishmaniasis with the conclusive clinical signs has a high level of antibodies mainly of the isotype IgG1, while an asymptomatic dog has antibodies specific to the isotype IgG2. The dogs that received our peptide complex have low levels of IgG2 specific to the A16E and/or A16G peptides, which is in keeping with the preferential expansion of T lymphocytes of the Th1 type.

The detection of the presence of IgG isotypes specific to the A16E and A16G peptides and their derivatives makes it possible, notably:
to show a humoral response depending on the Th 1 lymphocytes and thus to show an immune state of the Th1 type
to show a state of retarded hypersensitivity,
to monitor the immune response in vaccinated or treated mammals,
to monitor the effectiveness of a chemiotherapeutic and/or immunotherapeutic treatment.

The peptide complex comprising the A16E and A16G peptides and their derivatives and the adjuvant, according to the invention, can be administered in various ways. However, it is administered in a preferred manner in four ways:
either by subcutaneous injection
or by intradermal injection
or by intramuscular injection
or orally.

Other administration methods can be used, like the parenteral or intravenous method.

In a general manner, a vaccine appears in injectable form comprised of a lyophilizated fraction that is combined with a liquid fraction or diluent. The doses used for prevention and immunotherapy are different, and are also different depending on the mode of injection:
sub-cutaneous and intramuscular method:
injection of a dose (50 μg of peptides and 100 μg of adjuvant) in dogs regardless of breed and sex for a preventative effect).
injection of half doses (25 μg of peptides and 50 μg of adjuvant) for immunotherapy of leishmanian dogs.
intradermo method:
injection of a half dose in leishmanian dogs for a preventative effect
injection of a quarter dose in leishmanian dogs for a therapeutic effect.

The methods of injections are continued in the examples of immunotherapy and vaccination.

Immunotherapy Results

According to specialists such as PINELLI (PINELLI. E et al, Infect Immun, 1994, 62, 229-235), leishmanian dogs correspond to the activation of the lymphocytary system of the Th2 type having a high antibody response.

This increased production of antibodies corresponds to hyper-proteinemia and induces the appearance of immune complexes that cause a renal problem (increase in the creatinine and blood urea).

Thus, it was attempted to modulate towards a Th1 state by administering doses of the peptide complex by the intra-dermo method to the totally leishmanian dogs. The monitoring of the immune state and the clinical observation were done before and after treatment.

### EXAMPLE 1

### MANON, the Dog

A female dog of the British spaniel breed, age 4 years old, belonging to Mr. P, has numerous cutaneous lesions accompanied with a general state of fatigue and a thin appearance, all reminiscent of a leishmaniasic canine.

The veterinarian, Dr. L M, diagnoses leishmaniasis. This diagnosis is confirmed by a direct observation in a microscope of leishmanias from a cutaneous tracing and a serological analysis which gives a titer by immunofluorescence leishmaniasis positive at 1/3200.

The analysis of the immunitary state prior to any injection makes it possible to confirm that the dog was indeed in an immunitary state of the Th2 type with a high antibody titer as well as negative IDR tests and NO dosages. We established an immunotherapy which consisted in making 2 intradermo injections of 25 μg of peptides (½ A16E, ½ A16G) and 100 μg of muramyl dipeptide adjuvant, each injection being 3 weeks apart.

One week after the second injection, MANON the dog regained her appetite and a certain vitality. Dr. LM began to observe a slight cutaneous improvement.

One month after the last injection, MANON regained a normal clinical appearance with notably an increase in weight of 1 kg and a disappearance of 80% of all cutaneous lesions. Analysis of the immunitary state makes it possible to confirm a reduction in the anti-leishmania antibody titer which dropped to 1/400 by immunofluorescence. In parallel, the IDR (IDR performed with leishmaniasis and also with the A16E and A16G peptides), the NO dosage and the lymphoblastic proliferation were positive. In addition, after treatment, MANON the dog has IgG2 specific to the 2 peptides, IgG2 dosed by the ELISA method and Western Blot. These IgG2 specific were absent prior to any treatment.

A study of the parasites by cultivation on the NNN medium turned out to be negative. 8 months after treatment, MANON the dog did not show any modifications. The biological analyses make it possible to confirm that MANON is still in an immunitary state Th1.

### EXAMPLE 2

### PEPPONE the Dog

A male dog of the Griffon breed, named PEPPONE, age 5 years old, belonging to Mr. B, has clinical signs specific to leishmaniasis. According to Dr. GH, presence of numerous shiny squama, right periocular hair loss, ulcerous lesions at the level of the 2 front elbows, and a pronounced state of fatigue. Biological analyses with notably a positive leishmaniasis serology at 1/400 by immunofluorescence confirms the clinical diagnostic.

An immunotherapy was established, which consisted in making 3 intradermo injections of 25 μg of peptide complex (½ A16E, ½ A16G), and 100 μg of muramyl dipeptide adjuvant, each injection being 10 days apart. The analysis of the immune state prior to any injection showed that PEPPONE the dog had developed an immune system of the Th2 type with a greatly positive parasitemy from the bone marrow.

One month after the last injection, the leishmaniasic clinical signs of PEPPONE had retroceded with notably a healing of the ulcerous lesions, a sizeable disappearance of the squama and an almost non-existent periocular hair loss. The serology still has a titer by immunofluorescence equal to 1/400. On the contrary, the analysis of the cellular response makes it possible to confirm that PEPPONE has an active Th1 state with an IDR (IDR done with leishmanines and also with peptides A16E and A16G), positive dosage of NO and lymphoblastic proliferation.

In parallel, the parasitemy is negative (cultivation of the bone marrow in a NNN medium).

At the humoral level, PEPPONE the dog has IgG2 specific to the A16E and A16G peptides after treatment, IgG2 dosed with ELISA and Western Blot.

The present invention thus indeed consists of a therapeutic peptide complex that induces the passage from an immune state of the Th2 type, with sizeable production of antibodies that exacerbate the clinical manifestations, to an immune state of the Th1 type that leads to healing.

The peptide complex composed of two peptides A16E and A16G and the muramyl dipeptide adjuvant thus comprises a therapeutic compound.

Vaccination Results

In order to evaluate the efficacy of the peptide vaccine complex according to the invention, the vaccine complex was tested on 5 perfectly healthy dogs. These 5 dogs have a negative leishmaniasic serology, a negative parasitemy as well as fully negative cellular response tests specific to Leishmania. In addition, none of the 5 dogs has IgG2 specific to one or the 2 peptides A16E and/or A16G.

These 5 dogs live in a place free from any phlebotomes. We define 3 groups of dogs.
1—Control group (placebos)
   Negative control: the dog named LILI, Pointer breed, female, age: 3 years
   Sole adjuvant control: the dog named MIMI, British spaniel breed, female. Age: 6 years
2—Group of dogs vaccinated with peptides only (50 μg) and muramyl dipeptide adjuvant (100 μg)
   The dog named MAMA, Weimaraner breed, female. Age: 2 and a halfyears old =>peptide A16E
   The dog named NUNU, Pointer breed, male. Age: 2 and a half years old =>peptide A16G
3—Group of dogs vaccinated with peptides A16E and A16G (25 μgof each peptide) and muramyl dipeptide adjuvant (100 μg)
   The dog named LEON, British spaniel breed, male. Age: 4 years.

The vaccine injection scheme is as follows:

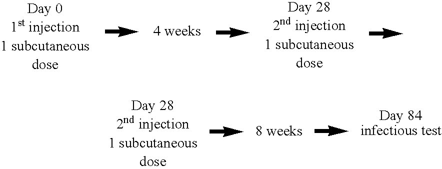

A clinical monitoring of the 5 dogs was done every two weeks. The biological analyses were scheduled as follows:

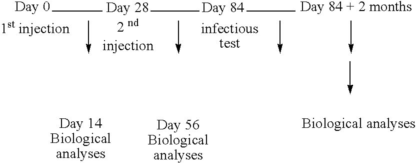

The biological analyses consist of:
biochemical analyses: urea, creatine, transaminases
hematological analyses: count, formula
serology leishmaniasis: quantitative anti-Leishmania immunofluorescence, dosage by the ELISA method of IgG2s specific to the peptides A16E and/or A16G
cellular response tests: IDR test (IntraDermoReaction) done with leishmanines and also with A16E and A16G peptides, dosage of NO and lymphoblastic proliferation.

To these analyses must be added the search for Leishmania by direct observation in a microscope and cultivation on NNN medium from bone marrow after the infectious test.

Results:

Clinical Monitoring:

No significant clinical manifestation appeared during all of this study. A slight weight loss and the appearance of some squama in the dog LILI must be noted, 4 months after the infectious test.

Biological Monitoring:
1—The biochemical and hematological parameters stayed normal all during this study.
2—Leishmaniasis serology and parasitemy.

Prior to any injection, the 5 dogs have negative serologies and parasitemies. The following table shows the serological responses obtained during our experiments and the monitoring of the parasitemy (analyses made 2 months and 12 months after the infectious test).

| | | SEROLOGY | | | Cultivation |
|---|---|---|---|---|---|
| | | IF | ELISA | PARASITEMY | on NNN |
| | Dogs | Quantitative | IgG2 | Direct Exam | Medium |
| Dogs | LILI | − | − | + | − |
| Control | MIMI | − | − | + | 0 |
| Dogs | MAMA (A16E) | − | +(0.700) | − | − |
| Immunized | NUNU (A16G) | − | +(0.520) | − | − |
| | LEON (A16E + | − | +(0.780) | − | − |
| | A16G) | | | | |

Key:
IF: Immunofluorescence (considered positive if the titer is ≧1/100)
ELISA: Cut off = 0.300 OD (optical density)
Parasitemy: cultivation on medium NNN
− = absence
++ = more than 5 mobile promastigote forms/field

Only the immunized dogs have specific antibodies of IgG2 (ELISA towards the corresponding peptides) and negative parasitemies. A slight appearance of total antibodies (1/200 in IF) must be noted in all of the dogs after the infectious test.

Only the control dogs (LILI and MIMI) have positive parasitemies and an absence of specific anti-peptide antibodies IgG2.

Cell-Mediated Response

Before any injection, the 5 dogs have a fully negative cell-mediated response to Leishmania infantum. According to the following table, only the immunized dogs have positive lymphoblastic proliferation tests, IDRs linked to the production of NO by the monocytes.

The following table shows the cellular type responses obtained (analyses made 2 months after the infectious test).

| | | | | | Dosage | Lymphoblastic |
|---|---|---|---|---|---|---|
| | | | IDR | | of NO | Proliferation |
| | Dogs | Leishmanines | A16E | A16G | (in μM) | Test |
| Dogs | LILI | + | − | − | 0, 3 | −1, 1 (3) |
| Control | MIMI | − | − | − | 0, 2 | −1, 2 (3, 1) |
| Dogs | MAMA (A16E) | + | + | Limit | 2, 6 | +2, 1 (3, 1) |
| Immunized | NUNU (A16G) | + | Limit | + | 2, 8 | ++3, 1 (3, 6) |
| | LEON (A16E + | + | + | + | 4, 2 | +++3, 7 (3, 8) |
| | A16G) | | | | | |

Key:
IDR: The Intra Dermo Reaction test is considered positive (+) if the induration is ≧5 mm 48 h. after intradermoinjection
Dosage of NO:
Lymphoblastic proliferation test: The results are expressed by a reading in a photon microscope and in stimulation indices (between parentheses, stimulation index + Concanavaline A)

From this analysis, the peptide compounds with the adjuvant do indeed induce a cell-mediated immunity of the Th1 protector type, to which it is necessary to add an induction of the antibodies of isotype IgG2. The mixture of the two peptides at an equal concentration thus gives a more pronounced cellular response (high rate of NO synthesized by activated monocytes).

The peptide complex comprised of the two peptides A16E and A16G and the muramyl dipeptide adjuvant constitutes a good vaccine complex.

1. Therapeutic peptide vaccine complex designed for prevention or treatment of conditions in mammals and in particular, in humans, canines, felidae, and equidae, whose protective immunity depends on the stimulation of lymphocytes of the type Th1 and notably of a retarded state of hypersensitivity, said therapeutic peptide vaccine complex comprising:
a sequence of amino acids (A16E): A-A-R-S-A-R-S-R-E-G-Y-S-L-T-D-E sequence in which L can be replaced by I, and S by C identified as SEQ. ID NO. 1;
a sequence of amino acids (A16G): A-A-S-S-T-P-S-P-G-S-G-C-E-V-D-G sequence in which C can be replaced by S, and S by C identified as SEQ. ID No.2; and
an adjuvant that preferably induces a cell-mediated response.

2. Therapeutic peptide vaccine complex according to claim 1, wherein the sequences A16E and A16G can be replaced by:
possible derivatives of the peptide A16E, these peptide derivatives comprising at least 5 adjoining amino acids from the sequence A16E
and/or possible derivatives of the peptide A16G, these peptide derivatives comprising at least 5 adjoining amino acids from the sequence A16G.

3. Therapeutic peptide vaccine complex according to claim 1, wherein the sequences A16E and A16G, or the derivatives of them, are preferably combined at the same concentration.

4. Therapeutic peptide vaccine complex according to claim 1, wherein said adjuvant is muramyl dipeptide.

5. Therapeutic peptide vaccine complex according to claim 4, wherein said muramyl dipeptide is combined with the peptides A16E, A16G or with their derivatives, in a ratio by weight peptide to adjuvant of 1/0.1 to 1/6.

6. Therapeutic peptide vaccine complex according to claim 4, wherein said muramyl dipeptide is combined with the peptides A16E, A16G or with their derivatives, in a ratio of 50 μg of proteins for 100 μg of muramyl dipeptide.

7. Therapeutic peptide vaccine complex according to claim 1, wherein the peptides A16E, A16G or their derivatives are constructed in octopus or are combined with large molecules of the KLH type or lipids, or included in the liposomes to make them immunogens.

8. Therapeutic peptide vaccine complex according to claim 1, being packaged in a form so that it can be administered by different methods: cutaneous, intradermal, intramuscular, intravenous, parenteral or oral.

9. Use of the vaccine complex according to claim 1, for the manufacture of a medicine, or of a vaccine or of a diagnostic reagent in vivo or in vitro for the induction or diagnosis in mammals of an activation of cell-mediated immunity depending on the T lymphocytes of the Th1 type.

10. Use of the vaccine complex according to claim 1, for the manufacture of a medicine, or of a vaccine or of a diagnostic reagent in vivo or in vitro for the induction or diagnosis in mammals of the passage from an immune state of the Th2 type to an immune state of the Th1 type.

11. Use of the vaccine complex according to claim 1, for the manufacture of a medicine, or of a vaccine or of a diagnostic reagent in vivo or in vitro for the induction or diagnosis in mammals of the isotypes of specific antibodies, such as the IgG2 in dogs, of cell-mediated immunity depending on the T lymphocytes of the Th1 type.

12. Diagnosis kit in vitro comprising the vaccine complex according to claim 1 further comprising large molecules of biotin or polylysine type in order to make them more antigenic.

13. Diagnosis kit in vitro comprising the vaccine complex according to claim 1, further comprising glutaraldehyde.

14. Diagnosis kit in vitro according to claim 12, wherein the peptides A16E and A16G, or their derivatives, are bonded with a solid support.

15. Diagnosis kit in vitro according to claim 14, wherein the peptides A16E and A16G, or their derivatives, are bonded with membranes of nitrocellulose or other polymers, latex supports and various plastic materials (polymer).

16. Diagnosis kit in vitro according to claim 12, wherein the peptides A16E and A16G, or their derivatives, are bonded with radio-isotopes, fluorescent molecules, luminescent molecules, enzymes and colored particles.
